(19) United States
(12) Patent Application Publication
(10) Pub. No.: US 2026/0045365 A1
(43) Pub. Date: Feb. 12, 2026

(54) DEVICES, METHODS AND ARTIFICIAL INTELLIGENCE SYSTEMS TO MONITOR AND IMPROVE PHYSICAL, MENTAL AND FINANCIAL HEALTH

(71) Applicant: Tutto Ltd, Coventry, West Midlands (GB)

(72) Inventor: Peter Arthur Luigi MAGNANI, Coventry, West Midlands (GB)

(73) Assignee: Tutto Ltd, Coventry, West Midlands (GB)

(21) Appl. No.: 19/100,963

(22) PCT Filed: Aug. 3, 2023
(86) PCT No.: PCT/GB2023/052062

§ 371 Date: Feb. 4, 2025

(30) Foreign Application Priority Data
Aug. 4, 2022 (GB) 2211386.4

(51) Int. Cl. G16H 50/30; G06N 3/063

(52) U.S. Cl. CPC G16H 50/30; G06N 3/063

(57) ABSTRACT

A system for monitoring data representative of a user's general wellbeing and for delivering digital content to the user, the system comprising a pre-defined module comprising a plurality of reference patterns mapped to digital content for delivery to the user, the system further comprising at least a processing means, wherein the processing means is configured to: identify user activity and select a user based on the identified user activity; for the selected user, interrogate user data representative of the user's general wellbeing and identify a pattern in said user data; and compare the identified pattern to the reference patterns in the pre-defined module to identify digital content mapped with the identified pattern.

### TECHNICAL FIELD

The present invention relates to a device to monitor and advise a person on their current health, and specifically their physical, mental and financial health, and well-being. The invention further relates to a method of monitoring said health and well-being. Specifically, the device and method can be delivered through a portable communication device such as a mobile telephone.

### BACKGROUND TO THE INVENTION

The importance of dealing with a person's mental well-being is increasingly recognised. In the UK, spending on mental health issues in general rose from £11 billion to £14.3 billion in the period from 2015/6 to 2020/1, and this figure is expected to increase, in spite of tightening economic conditions. There is also an increasing recognition this state can play as important a role in a person's performance or attendance at work as what are often referred to as the person's physical, mental and financial well-being. According to a joint release in 2021 by the “World Federation for Mental Health” and the “World Health Organisation”, generalised anxiety is one of the most neglected areas of public and private services worldwide. Close to 1 billion people are living with everyday stress-related issues and its physical and financial side-effects, yet relatively few have access to effective and ongoing support to help them eliminate unnecessary stress, better manage necessary stress and prevent escalation to clinical conditions.

Although the awareness of mental well-being is increasing in some countries, there remains difficulty in accessing help to deal with its causes and effects. State funding for healthcare is becoming increasingly stretched in many countries and the ability to provide assistance in this area is often one of the first to be reduced.

There is therefore an increased need to provide readily accessible assistance to people to both deal with the causes of lack of mental well-being to try and also to prevent a negative state of physical, mental, and financial well-being from developing. Analysing data reflective of a user's physical, mental, and financial well-being state requires significant labour cost, because it can only typically be carried out by highly qualified personnel across a number of disciplines, including medical personnel.

The present invention seeks to address the above problem through the provision of a means of preventing and/or determining a person's mental state and enabling action to be taken to reduce the effects thereof and move a person into a healthier state.

Specifically, an aim of the present invention is to provide a system and method that is capable of automatically and quickly assess user data corresponding to a physical, mental, and financial well-being state in order to determine the risk of developing a negative state.

### SUMMARY OF THE INVENTION

According to a first aspect of the invention, there is provided a system for monitoring data representative of a user's general wellbeing and for delivering digital content to the user, the system comprising a pre-defined module comprising a plurality of reference patterns mapped to digital content for delivery to the user, the system further comprising at least a processing means, wherein the processing means is configured to:
identify user activity and select a user based on the identified user activity;
for the selected user, interrogate user data representative of the user's general wellbeing and identify a pattern in said user data; and
compare the identified pattern to the plurality of reference patterns in the pre-defined module to identify digital content mapped with the identified pattern, for delivery to the user.

The pre-defined module is referred to as a categorisation and taxonomy module which maps relevant content to identified user behaviours identified from the patterns. The digital content may be digital media content. Preferably, the digital content comprises user guidance which can be in the form of, but not limited to, video, written, audio and content solutions from the internet.

In a dependent aspect, the system further comprises a user interface configured to deliver the identified digital content to the user.

In a dependent aspect, the step of identifying a pattern comprises the sub-steps of: providing a plurality of pattern indicators for a selected time period;
selecting a sub-set of pattern indicators from the plurality of pattern indicators; and using a neural network to identify the pattern using the selected sub-set.

In a dependent aspect, the processing is further configured to determine a plurality of user score categories, comprising the sub-steps of:
identifying a plurality of pattern indicators corresponding to a score category;
calculating average values of the plurality of pattern indicators over a selected period of time and comparing the respective last calculated average values when the method steps are repeated, to thereby identify a change in said values;
determine whether said change has a positive or negative impact on the well-being of the user, to provide multiplier values.

Advantageously, each time the artificial intelligence system is run, new patterns identified may be processed by comparison against those already existing on the database for the particular user and which have been delivered to the user.

According to a second aspect of the invention, there is provided a system for training artificial intelligence in order to process data representative of a user's general wellbeing, the system comprising a pre-defined categorisation and taxonomy module comprising a plurality of reference patterns mapped to digital content for delivery to the user, the system further comprising at least one processing means, wherein the processing means is configured to:
receive a training data set including a plurality of labelled data entries representative of the user's general wellbeing, and
   perform the following sub-steps, until a predetermined condition is met, preferably a number of epochs is reached, or a configured threshold of accuracy is reached:
   identify a pattern in the labelled data entries;
   compare the identified pattern with a reference pattern of the pre-defined categorisation and taxonomy module;
      optimise weights and biases of the neural network based on the comparison result;
      update the weights and biases of the neural network; and
   generate a neural network configuration comprising the updated neural network weights and biases, when the predetermined condition is met.

Thus, a system according to the invention may comprise one or more processing means, each of the processing means being able to execute one or more of the above steps. Also, the processing means can be run on one or more computers, which could be standalone workstations or virtual servers in a cloud environment.

According to a third aspect of the invention, there is provided a method for monitoring data representative of a user's general wellbeing and for delivering digital content to the user, the method comprising the steps of:
providing a pre-defined module comprising a plurality of reference patterns mapped to digital content for delivery to the user;
identifying user activity and selecting a user based on the identified user activity;
for the selected user, interrogating user data representative of the user's general wellbeing and identifying a pattern in said user data; and
comparing the identified pattern to the plurality of reference patterns in the pre-defined module to identify digital content mapped with the identified pattern, for delivery to the user.

According to a fourth aspect of the invention, there is provided a method of training artificial intelligence in order to process data representative of a user's general wellbeing, the method comprising the steps of:
providing a pre-defined categorisation and taxonomy module comprising a plurality of reference patterns mapped to digital content for delivery to the user;
receiving a training data set including a plurality of labelled data entries representative of the user's general wellbeing, and
   performing the following sub-steps, until a predetermined condition is met, preferably a number of epochs is reached or a configured threshold of accuracy is reached:
   identify a pattern in the labelled data entries;
   compare the identified pattern with a reference pattern of the pre-defined categorisation and taxonomy module;
      optimise weights and biases of the neural network based on the comparison result;
      update the weights and biases of the neural network; and
   generate a neural network configuration comprising the updated neural network weights and biases, when the predetermined condition is met.

All the expressions used in the description of system for training artificial intelligence have the same meaning as the expressions used in the description referring to machine learning algorithms and artificial intelligence.

Aspects of the invention have technical advantages over known solutions, being capable of automatically and quickly assessing user data corresponding to a physical, mental and financial well-being state in order to determine the risk of developing a negative state. In addition, known digital health systems are a “one size fits all” solution, use delayed insight and are predicated on an advertising or data aggregation business model, not real human needs. The main established systems are also siloed (focusing on one aspect of life e.g. exercise, sleep, meditation etc.) whereas each person is unique, multi-dimensional and capable of solving many of their problems with the right tools. Aspects of the invention described above assess relevant user data in real time, for effectively and quickly achieving said reduction in an individual's problems or the effect of those problems.

Further advantageously, the present invention allows a user suffering stress to intervene on their own behalf. In preferred embodiments, the system is implemented in the form of an app, maintained on a person's mobile telephone or the like, which measures, tracks and interprets aspects of daily living to identify the behaviours, habits and routines people need, in order to minimize unnecessary stress and boost resilience. The trained artificial intelligence system provides an evidence-based methodology and is built on the foundational principle that mental, physical and, crucially, financial well-being are intrinsically linked. The system functions by securely integrating a user's personal data with algorithms, ontologies and analytics to quantify, decode and improve a user's life. A holistic product, which links a user's health and financial data automatically and quickly is thus provided.

### BRIEF DESCRIPTION OF THE FIGURES

The invention is described with respect to the accompanying Figures which show by way of example only, an embodiment of a system to aid in the determination and management of mental well-being. In the drawings:

FIG. 1 is a general overview of elements of the system;

FIG. 2 is a flow-chart showing the flow of data within the system;

FIG. 3 illustrates pattern-identification processes;

FIG. 4 illustrates a quantification process and metric used in the system; and

FIG. 5 illustrates a data-scoring methodology.

### DETAILED DESCRIPTION OF THE INVENTION

In broad overview, a 3-part system is disclosed. In the first part, a user's emotional triggers and behavioural patterns are identified through the use of pre-set algorithms and machine learning routines to analyse the user's behaviour. In general, correlations and statistically relevant relationships between data points are identified. These data points may cover mood, health/wearable sensors data, bank spend across categories, screen time across categories, weather and location etc. In the second part, the system automatically analyses this data, in real time and identifies using, a logic weighting algorithm to aggregate and analyse the data, the relationship between these collected behaviours and the feelings of each individual user. In the third part, the behaviours that are most influential on overall well-being are then highlighted, both positive and negative, and how they inter-relate with the thoughts and actions people take.

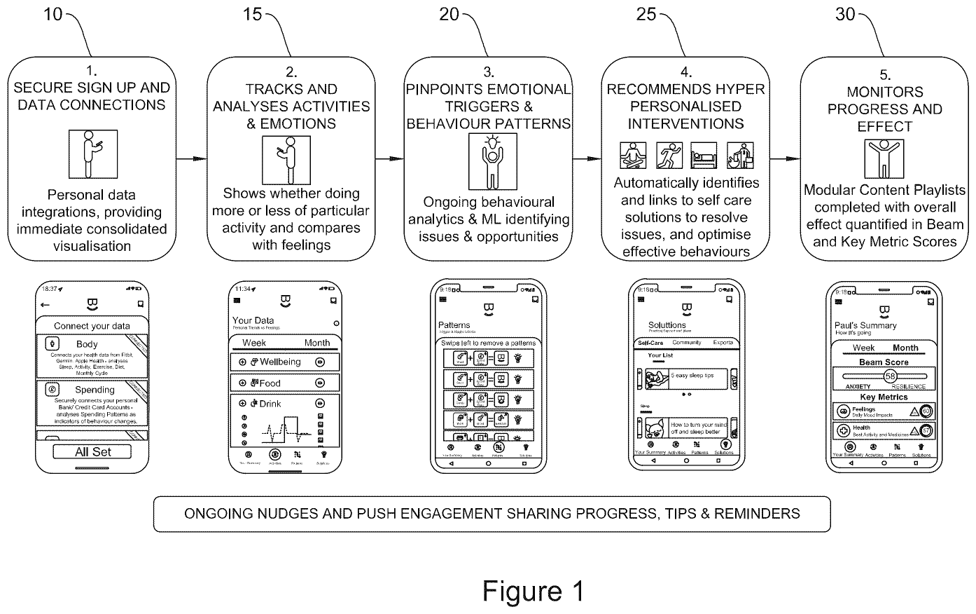

Referring now to FIG. 1, this provides an overview of the system disclosed herein. In summary, five separate modules relating to stages of a process can be defined. In the first stage indicated at 10, the user signs up to the system. Personal data is obtained from the user which can then be processed utilising an algorithm and based around a machine-learning process. The analysis carried out supplies a time-dependent snapshot of the user's current mental well-being status and an insight into things or events which are important to the user or which change that status: whether positively or negatively.

In the second stage 15, the user is monitored using a defined set of input data comprising activities undertaken and also user input information, for example relating to their well-being status. The system utilises the data obtained to determine which activities have a particular effect on the user, in the third stage 20, in which on-going behavioural analytics and machine-learning identify relevant issues and opportunities. The issues identified in the third stage 20 are addressed in the fourth stage 25 in which the system identifies self-care solutions to negative issues and directs the user to said solutions in order to optimise their effective behaviours. This can be in the form of nudges, push-engagement, sharing progress, tips and reminders. In the fifth stage 30, the progress of the user is monitored, and various aspects of the user's behaviour are assigned a quantifiable value, utilising a defined metric. This allows the progress or otherwise of a user to be kept under observation by the user.

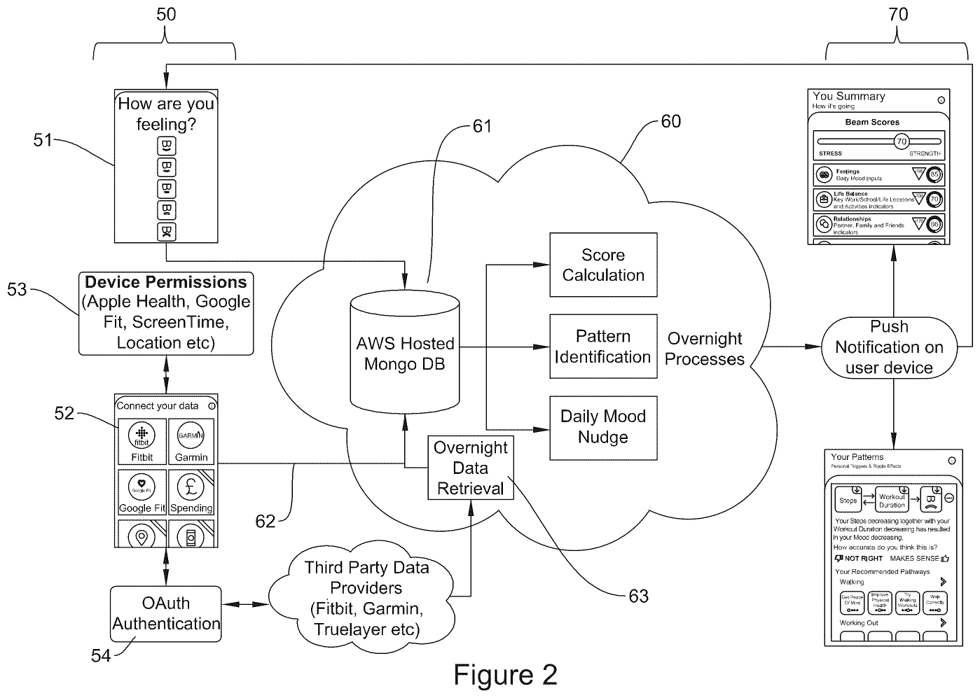

A more detailed view of data flow within the system is given in FIG. 2. In a broad overview, data 50 is obtained from the user, including access rights to the user's information relating to financial transactions and the like. This data 50 is fed into the processor 60 which processes the data 50 to produce a determination, preferably in a quantifiable form relating to the state of the user's well-being. The output is sent to the user 70 in readily interpretable form on a device of their choosing.

The data obtained and processed relating to a user is taken from many different sources in order to gain a fuller picture of the user regarding their mood, habits, drives etc. As financial issues are known to be an influence on the well-being of many people, the data includes spending transactions and patterns which can be tied in to a person's state of mind. Amongst the data 50 are shown, as non-limiting examples, data sets which are preferably included within the data obtention step. For example, at 51, a user input assessment of the user's current mental state is obtained. This can be a simple icon-selection step using readily recognisable emojis, or can include a more detailed set of questions put to the user. In this step, thousands of data points can be brought from the user. Sensor data 52 is also gathered from devices such as a Garmin (RTM), Fitbit (RTM), spending information, location, and other devices etc. A part of this step will therefore require software processing methods to be located on the user's device either enabling the user to input the data or to allow the data gathering to occur in the background. Permissions are granted 53 by the user to enable this to be carried out. The information is passed after authentication 54 to the processor 60.

It is important that security of such data is ensured as much as possible. In an example, all connections to a user's data sources are provided in a secure manner using OAuth™ technology which is required to authenticate a user via either a https web connection or through third party apps installed on a user's device. The token which results from this is passed to the backend (see below) for storage and use during data synchronisation. This is also used for any Webhooks which may be required from time to time, for example to receive real time updates.

With regard to the backend, a processor 60, in a preferred embodiment, is housed on the Amazon Web Services (AWS) Cloud Engine, and would not normally be accessible to the user. The data obtained from 50 is stored on a MongoDB database 61, which can be updated either in real-time 62 from the user, or from overnight data retrieval from an intermediate database 63. An analysis algorithm performs calculations on the data and assigns one or more scores for the user. In determining the scores, the algorithm takes into account that different information sources are available for each user and calculations and weightings take this into account. It is important that consistent value assignments are made so that the results can be relied upon and are not unduly influenced by a particular data source or number of times a data source is accessed or by the frequency with which a user inputs data relating to their own feelings. In one embodiment, the value assignments are checked by data scientists although other methodologies known in the art can be utilised. In one embodiment, the values are assigned manually against multiple datasets. A validity filter can be used comprising 3 core elements: modelled norms, industry benchmarks and subjective sense checking.

Once processed an Application Programming Interface (API) allows the output values to be displayed 70 on the device selected by the user. To assist the broad applicability, Flutter is used as the application framework to allow simplified development of software for both iOS and Android, thus reducing implementation time for any new features. Some code is still required on each platform to enable the information from advanced technologies such as Apple Health and Google Fit, and location tracking using Geofences. The information provided can be an overall summary, for example in the form of a single number illustrating the user's well-being, or can also provide more detailed information, such as patterns of behaviour which a user might wish to address.

In a further aspect of the invention, once a pattern of behaviour has been identified, a category and taxonomy builder is utilised to obtain possible guidance aids to the user, preferably in real time, to assist the user to change that behaviour or learn to cope with the behaviour. The category and taxonomy builder also enables digital content of particular relevance to be delineated and extracted. The extracted material ensures that the right content is provided to a user with minimal superfluous material. Any guidance data, once identified, can be provided to the user, typically via pathways containing 4-part modules of practical tips and pertinent advice. The guidance can be in the form, but not limited to, video, written, audio and content solutions from the internet.

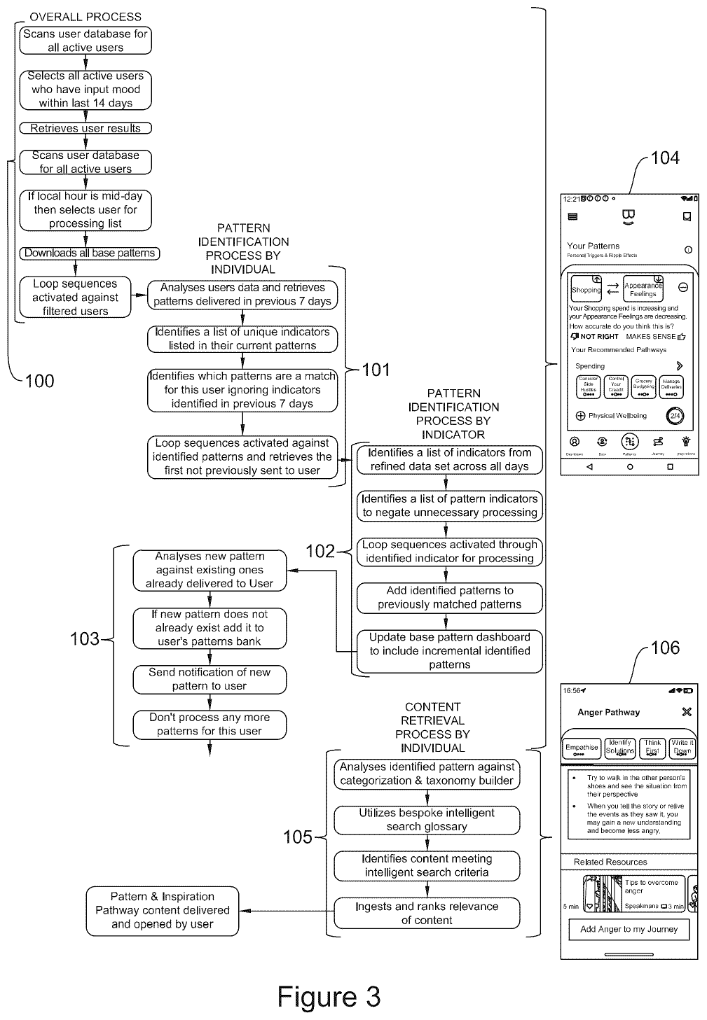

An example of steps undertaken by the device and method utilised is given in FIG. 3 which illustrates a method of handling multiple users, including pattern recognition and content selection. Beginning with the user selection, in section 100, the user database is first scanned to determine the users who are active. In an example, where a user has input some data relating to their mood within the previous 14 days, then the results for those users, such as name, e-mail, messaging, tokens etc., are accessed from the users' table. Should the local time for a particular user be close to a pre-set value, for example midday, then the user is added to a processing list and the base patterns identified by machine-learning algorithms analyses are downloaded, including Loop sequences which analyse all identified users against base patterns are then activated against the selected users and these users passed to a pattern identification algorithm 101. In an example, the patterns delivered in the previous 7 days to these selected users are retrieved and a list of unique identifiers in the current patterns of these users identified. The patterns are identified for this user which are a match, ignoring those identified in the previous 7 days, and loop sequences activated against identified patterns: the first pattern identified, not previously sent to the user is retrieved.

In section 102, the pattern identification process is exemplified. First, a list is identified of indicators selected from a refined data set across all days in a selected time period, for example a selected period of 30 days. Pattern indicators are then identified to minimise unnecessary processing, and loop sequences activated through the identified indicator for processing. The patterns identified here are added to previously matched patterns. The base pattern dashboard is then updated to include the additional identified patterns. The new patterns identified are processed 103 by comparison against those already existing on the database for the particular user and which have been delivered to the user. New patterns are then added to the user's database of patterns, also referred to as the pattern bank. A notification 104 of the new pattern is sent to the user and the profile of the user updated to indicate that no further patterns should be sent to the user within a predefined time period.

In order for a user to retrieve content identified 105, the identified pattern is identified against the category and taxonomy builder. This employs a pre-set intelligent search glossary and then identifies content which matches the criteria of the search. The relevance of a particular document or media content is ranked. Data is obtained from the internet covering scientifically validated physical, mental and financial health solutions and approved Points of View. This is then processed to obtain answers and insights. A pre-defined categorisation and taxonomy builder is used to filter by understanding the relevance of content by creating a single unified search index to allow for the homogenous ranking of search results, regardless of their source. Content is analysed to recognise the videos, articles and audio and classify them, identifying the correlations between the individual pieces of content and the categorisation. Personalised recommendations are then provided to users. The identified document or media content is despatched 106 to the user.

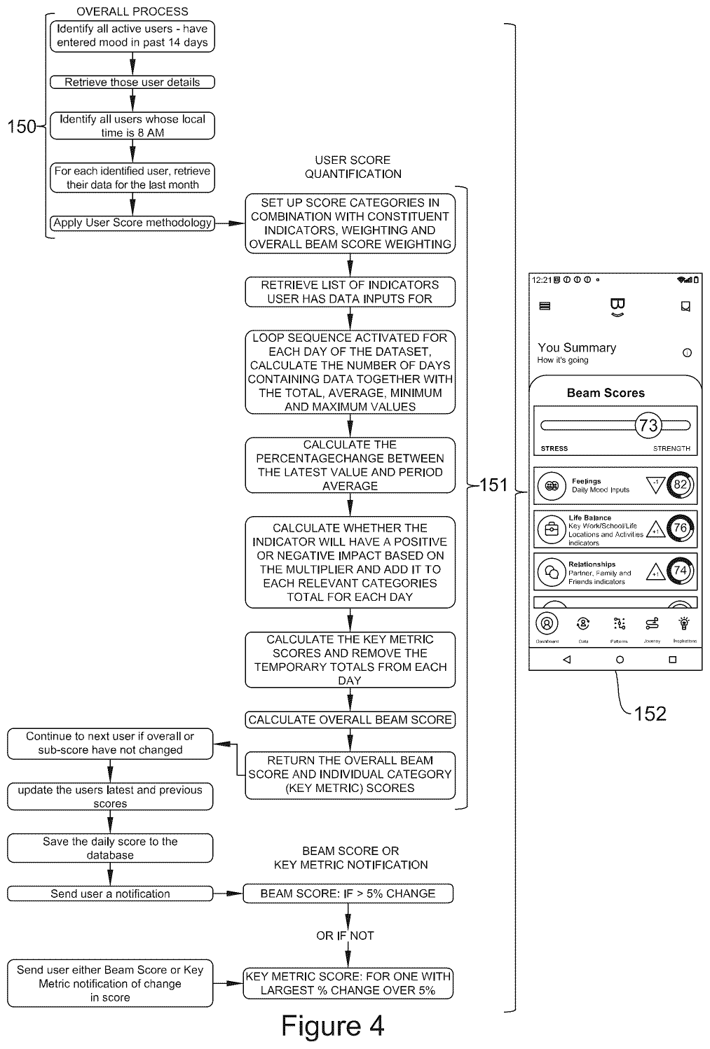

A method of quantifying characteristics of a user is outlined in FIG. 4. Firstly, in section 150, the users to be processed are identified and their data retrieved, optionally the data relates to the previous month. In the exemplified method of FIG. 4, all active users who have entered a mood rating within the previous 14 days are the ones identified, although different time periods can be chosen if so required.

The data is then quantified following the process 151 shown. Score categories are set up in combination with constituent indicators, weighting and overall score rating. The list of indicators for which a user has data inputs is retrieved, by running a query against all the data to see if a user has data for each indicator. A loop sequence is activated for each day of the dataset. The number of days of the dataset containing data is determined along with the total, minimum, maximum and average values of each Indicator. The percentage change between a latest value and the period average is determined and this value compared with a comparison dataset to determine whether the indicator is expected to have a positive or a negative impact on the user, based on the multiplier. The multiplier can be either +1 for positive or −1 for negative. The indicator's value is then added to relevant categories' totals for each day according to the weighting assigned to that indicator. The key metric scores are calculated and any temporary totals removed from each day, allowing the overall score for the user to be calculated.

Once the above steps have been carried out, then if the scores are unchanged for a user, the next user is selected. Otherwise, the user's latest and previous scores are updated, and the daily score saved to the database. The score is sent to the user 152.

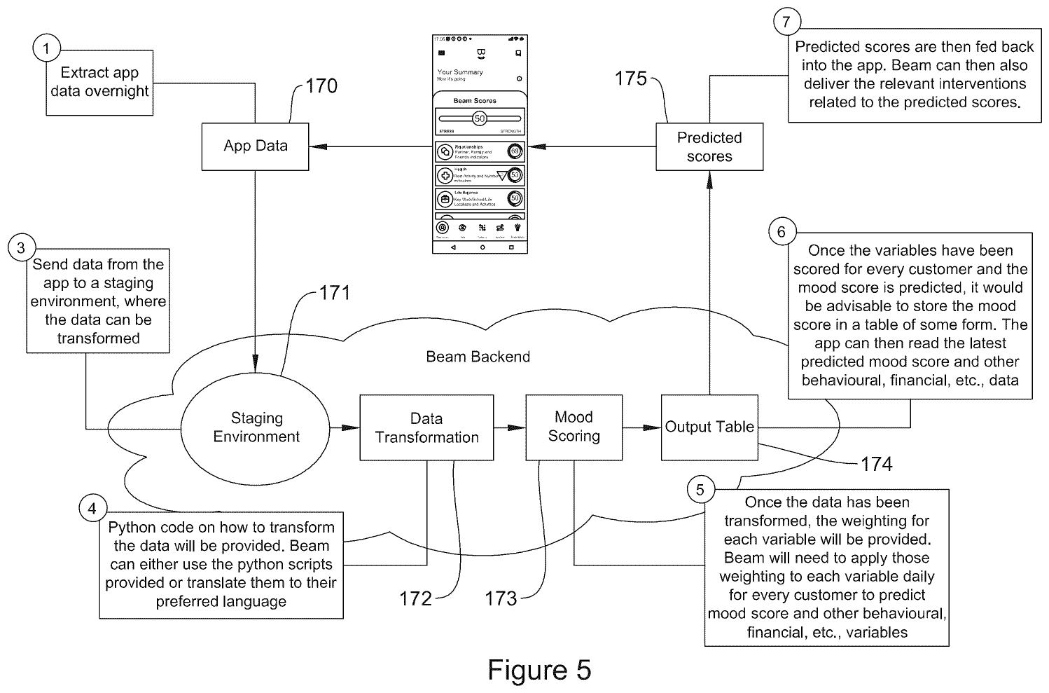

FIG. 5 illustrates a methodology utilised to analyse data and to assign a value to the wellbeing status of a user. In a first stage 170, data is downloaded, for example from an app which is typically located on a user's smartphone. Optionally the data is downloaded during the night to allow for efficient usage of a user's data bandwidth. The data obtained is sent to a staging environment 171 in which the data is transformed into a usable form. Optionally, Python code can be used to transform the data, or a suitable compiler to translate into a coding language of choice. Once the data is transformed 172, the transformed data is passed to a mood scoring module 173. The weighting value for each variable identified is provided. This weighting is preferably carried out on a daily basis for a user to provide a finer view of a user's wellbeing. The weighting value is utilised to predict a user's mood score and also assign a score to other aspect of a user's life such as behaviour, financial status etc.

When values have been assigned to each variable and the mood score has been predicted, then the mood score is preferably tabulated in a table 174. The device is enabled to interrogate the table to obtain the most up-to-date mood score along with the other behaviour, financial status data. The predicted scores 175 are fed back into the app along with any interventions identified as required or helpful.

1. A system for monitoring data representative of a user's general wellbeing and for delivering digital content to the user, the system comprising a pre-defined module comprising a plurality of reference patterns mapped to digital content for delivery to the user, the system further comprising at least a processing means, wherein the processing means is configured to:
identify user activity and select a user based on the identified user activity;
for the selected user, interrogate user data representative of the user's general wellbeing and identify a pattern in said user data; and
compare the identified pattern to the plurality of reference patterns in the pre-defined module to identify digital content mapped with the identified pattern.

2. A system according to claim 1, further comprising a user interface configured to deliver the identified digital content to the user.

3. A system according to claim 1, wherein the step of identifying a pattern comprises the sub-steps of:
providing a plurality of pattern indicators for a selected time period;
selecting a sub-set of pattern indicators from the plurality of pattern indicators; and
using a neural network to identify the pattern using the selected sub-set.

4. A system according to claim 3, wherein the processing is further configured to determine a plurality of user score categories, comprising the sub-steps of:
identify a plurality of pattern indicators corresponding to a score category;
calculate average values of the plurality of pattern indicators over a selected period of time and compare the respective last calculated average values when the method steps are repeated, to thereby identify a change in said values; and
determine whether said change has a positive or negative impact on the well-being of the user, to provide multiplier values.

5. A system for training artificial intelligence in order to process data representative of a user's general wellbeing, the system comprising a pre-defined categorisation and taxonomy module comprising a plurality of reference patterns mapped to digital content for delivery to the user, the system further comprising at least one processing means, wherein the processing means is configured to:
receive a training data set including a plurality of labelled data entries representative of the user's general wellbeing, and
perform the following sub-steps, until a predetermined condition is met, preferably a number of epochs is reached or a configured threshold of accuracy is reached:
identify a pattern in the labelled data entries;
compare the identified pattern with a reference pattern of the pre-defined categorisation and taxonomy module;
optimise weights and biases of the neural network based on the comparison result;
update the weights and biases of the neural network; and
generate a neural network configuration comprising the updated neural network weights and biases, when the predetermined condition is met.

6. A method for monitoring data representative of a user's general wellbeing and for delivering digital content to the user, the method comprising the steps of:
providing a pre-defined module comprising a plurality of reference patterns mapped to digital content for delivery to the user;
identifying user activity and selecting a user based on the identified user activity;
for the selected user, interrogating user data representative of the user's general wellbeing and identifying a pattern in said user data; and
comparing the identified pattern to the plurality of reference patterns in the pre-defined module to identify digital content mapped with the identified pattern.

7. A method of training artificial intelligence using a system according to claim 5.
